(19) United States
(12) Patent Application Publication
(10) Pub. No.: US 2005/0118055 A1
(43) Pub. Date: Jun. 2, 2005

(54) Chemical compositions and methods of absorbing moisture and odour from a chamber

(76) Inventor: David Bedford, Hull (GB)

Correspondence Address: NORRIS, MCLAUGHLIN & MARCUS, 875 THIRD AVE, 18TH FLOOR, NEW YORK, NY, 10022, US

(21) Appl. No.: 10/502,724

(22) PCT Filed: Jan. 29, 2003
(86) PCT No.: PCT/GB03/00360

(30) Foreign Application Priority Data
Jan. 30, 2002 (GB) 0202061.8

(51) Int. Cl. A61L 9/014; A61L 11/00

(52) U.S. Cl. 422/5; 424/76.5

(57) ABSTRACT

The invention relates to a method of absorbing moisture and odour from a chamber, for example a waste receptacle. This is achieved by addition to the chamber of a desiccant composition comprising a substantially water-insoluble polyacrylate compound capable of absorbing at least its own weight of moisture, and an odour-combating zeolitic material.

This invention relates to the use of desiccant compositions in absorbing moisture and malodour from the interior of chambers, for example waste receptacles for moist and malodorous waste materials.

It is known to provide odour absorbing and/or sterilizing compositions for use in combating odour produced by domestic waste and by bodily fluids. In particular, domestic waste placed in bins and waste receptacles can generate significant malodour if left for any period of time, especially when the waste is organic such as food and beverage waste. Part of the malodour may be formed by gaseous compounds released from the waste material, and part of the malodour may be formed by volatile compounds within moisture present in the waste material.

Various odour controlling agents have been disclosed in the literature. Many odour-control materials have been described for use with sanitary articles such as nappies and feminine hygiene bins. U.S. Pat. No. 5,885,263 discloses compositions comprising super absorbent polymers containing boron species, which absorb moisture from waste material in the vicinity of the odour controlling composition. WO 01/52912 discloses absorbent articles, such as sanitary napkins, panty liners and nappies comprising lactic acid producing micro-organisms and odour controlling zeolite compounds which absorb malodours from waste material. WO 91/11977 discloses the use of zeolites having intermediate SiO₂/AlO₂ ratios to control odours in sanitary articles such as nappies and panty liners.

One problem with the odour-control compositions disclosed in the prior art discussed hereinabove, is that each of the compositions comprises either a moisture absorbing agent of limited moisture absorbing ability, in conjunction with a malodour absorbing agent, or comprises a super absorbent moisture absorbing agent without a malodour absorbing agent. Thus, in each case, only a limited quantity of both moisture and malodour may be absorbed by the compositions described, and they are therefore disadvantaged in relation to combating odour from organic waste material commonly present in domestic waste bins. Thus, the prior art compositions generally either absorb large quantities of moisture but small quantities of malodour in an airspace in which the compositions are placed, or absorb malodour in the airspace, but small quantities of moisture from the waste.

It would therefore be advantageous to provide a composition which absorbed both malodour and large quantities of moisture from a wide range of waste materials.

According to a first aspect of the present invention there is provided a method of absorbing moisture and combating odour in the interior of a chamber containing moist material, the method comprising the step of adding to the chamber a desiccant composition in a powder or granular form or as a tablet or block which falls in use into a powder or granular form, the desiccant composition comprising a substantially water-insoluble polyacrylate compound capable of absorbing at least its own weight of moisture from the moist material in the chamber, and an odour-combating zeolitic material.

The term “desiccant composition” is used throughout this specification to denote the composition defined above, having moisture absorbing and odour-combating properties.

Suitably the compound capable of absorbing at least its own weight of moisture is a compound capable of absorbing at least ten times its own weight of moisture, preferably at least thirty times its own weight of moisture and more preferably at least fifty times its own weight of moisture. The composition is made with the compound in a desiccated condition.

Preferably the compound capable of absorbing at least its own weight of moisture is a compound with a high capacity to absorb water in its vicinity. Thus, it preferably has a high capacity to absorb standing water and water entrained in waste materials, for example food waste and sanitary materials. Its facility to absorb water vapour is of minor relevance, in this invention.

The compound capable of absorbing at least its own weight of moisture is a substantially water insoluble polyacrylate, preferably sodium polyacrylate.

Such polymers may comprise the entire absorbent compound or may be grafted onto other types of polymer moieties such as starch or cellulose.

Suitably the compound capable of absorbing at least its own weight of moisture is present in an amount of at least 0.1 wt %, preferably at least 0.5 wt %, and most preferably at least 1 wt %, of the weight of the dry composition. Suitably the compound capable of absorbing at least its own weight of moisture is present in an amount of no more than 25 wt %, preferably no more than 20 wt %, most preferably not more than 10 wt % and, especially, not more than 5 wt % of the weight of the dry composition.

The term “odour-combating” in this specification refers to any manner in which odour is counteracted by way of a physical or chemical action, for example by absorption, suppression, neutralisation or degradation; not merely odour-masking, as a fragrance alone may achieve.

Suitably the odour-combating zeolitic material is present in an amount of at least 0.1 wt % of the weight of the dry composition, more preferably at least 0.5 wt %, most preferably at least 1 wt %. Suitably the odour-combating zeolitic material is present in an amount of no more than 25 wt %, preferably no more than 20 wt %, and most preferably no more than 10 wt % of the weight of the dry composition.

Preferably the odour-combating zeolitic material is a flowable zeolitic powder.

Suitably an odour-combating zeolitic material, which is preferably a flowable zeolitic powder, comprises at least 80 wt % zeolite, and preferably at least 90 wt % zeolite. It may in certain useful embodiments be constituted substantially entirely by zeolite. When it is not, it may be zeolite admixed with a filler or, preferably, processing aid.

In this specification references to “zeolite” are to substantially virgin zeolite, including bound water of crystallisation and any atmospherically absorbed unbound water that may be present, but not including deliberately added materials. “Zeolitic material” denotes zeolite (as just defined) and also such a material containing deliberately added material(s).

Definitions which refer to the weight or weight ratio of zeolitic material are made with reference to the zeolite (as defined above) in the zeolitic material, unless otherwise stated.

We believe that the invention can be applied using any type of odour-absorbing zeolitic material, including odour-absorbing grades of the materials known as zeolite MAP, zeolite X, zeolite P and, most preferably zeolite A.

Preferably the composition further comprises a filler.

Suitably the filler is an inorganic filler and preferably is an inorganic salt filler. The filler may be a processing aid and may itself have water-absorbing properties.

By “filler” we mean a compound which serves to increase the bulk of the composition and which, preferably, is substantially water insoluble.

Suitable fillers include, for example calcium carbonate, cellulose or cellulose derivatives, anhydrous calcium sulphate, calcium oxide, silica gel, and bentonite clays.

Especially preferred as a filler is calcium carbonate.

Suitably the filler is present in the composition in an amount of at least 50 wt %, preferably at least 60 wt %, more preferably at least 75 wt % and most preferably at least 85% of the weight of the dry composition.

Suitably the filler is present in the composition in an amount of no more than 99 wt %, preferably no more than 98 wt %, more preferably no more than 95 wt %, of the weight of the dry composition.

Some compounds which are primarily present as a filler may have odour-combating properties. Suitable fillers which have odour-combating properties include inorganic carbonates such as calcium carbonate, clays such as bentonite clays and silica gel.

However, preferred compositions comprise both highly effective odour-combating compound and a separate filler which may or may not also absorb some odour.

Even though some fillers also have some odour-combating properties, in this invention a filler is used in conjunction with a separate odour-combating compound, and thus is intended primarily as a bulking material, and thus is not an “odour-combating compound” according to the invention.

A preferred composition of the invention comprises a polyacrylate compound capable of absorbing at least its own weight of moisture, a filler, especially calcium carbonate, and an odour-combating zeolitic material.

The desiccant composition may, as a component additional to the odour-combating compound, further comprise a fragrance. Many suitable fragrances are commercially available, including those designed to mask waste odours, and the choice of fragrance is a matter of subjective choice.

Advantageously, the composition is in any suitable solid form, such as a tablet, block, powder, or granules. A tablet or block falls in use into a powder or granules. Preferably, however the composition is supplied in the form of a powder or granules.

Suitably the chamber mentioned above is the interior of a waste receptacle. Preferably it is a substantially enclosed waste receptacle.

Suitably the method comprises providing a waste receptacle into which waste material is to be subsequently deposited, and adding a layer of desiccant composition onto the bottom of the waste receptacle. Suitably the layer of desiccant material is added to the bottom of the waste receptacle before waste material is deposited therein. Alternatively or additionally the desiccant composition may be added to waste material already deposited in the waste receptacle. Advantageously there may be at least two layers of waste material and at least two layers of desiccant, in the waste receptacle.

Suitably the waste receptacle is a refuse bin.

In accordance with a further aspect of the present invention there is provided a composition comprising:
(a) 0.1-25 wt % of a substantially water insoluble polyacrylate compound capable of absorbing at least its own weight of moisture from moist material it is in contact with;
(b) 0.1-25 wt % of an odour-combating zeolitic material;
(c) at least 50 wt % of filler;
the composition being particulate or a solid composition which falls into a particulate form in use.

In accordance with a further aspect of the present invention there is provided a product comprising a flowable particulate composition comprising a substantially water insoluble polyacrylate compound capable of absorbing at least its own weight in water and an odour-combating zeolitic material; the product comprising a container for the composition, the container having a dispenser therefor.

The dispenser may comprise an opening under the control of a user. The user exposes the opening when dispensing is required. There may be a plurality of openings, to act as a sprinkler head.

### EXAMPLE

The invention will now be described by way of the following, non-limiting example, in which the following materials are used:
Sodium polyacrylate: Alcosorb G3 from Ciba Speciality Chemicals, UK, a sodium polyacrylate capable of absorbing approximately 60 times its own weight of water.
Zeolitic material: Zeocros E100 powder from Ineos Silicas, UK.
Calcium carbonate filler: Minfil 400 calcium carbonate powder supplied by OMYA PLC, UK.
Fragrance: powder form, as desired.

### Example 1

A desiccant composition for use in the method of the invention was prepared by mixing the following ingredients in a standard tumble mixer to yield a desiccant powder:

| Component | Amount w/w |
|---|---|
| Calcium carbonate filler | 93.75% |
| Sodium polyacrylate | 1% |
| Zeolitic material | 5% |
| Fragrance | 0.25% |

A thin layer of the desiccant composition was sprinkled onto the base of an unlined, lidded kitchen bin. A 50 mm layer of vegetable waste was added, followed by successive thin layers of desiccant composition and further vegetable waste until the bin was full. An identical control bin was filled with identical vegetable waste but with no desiccant composition. The control bin soon became malodorous and the vegetable waste seeped free water which collected around the waste, at the bottom of the bin. The bin with the layers of desiccant composition took several days to become malodorous and did not to that point seep water.

1. A method of absorbing moisture and combating odour in the interior of a chamber containing moist material, the method comprising the step of adding to the chamber a desiccant composition supplied in the form of a flowable powder or granules or as a tablet or block which falls in use into a powder or granular form, the desiccant composition comprising a substantially water-insoluble polyacrylate compound capable of absorbing at least its own weight of moisture from the moist material in the chamber, and an odour-combating zeolitic material.

2. A method according to claim 1, wherein the compound capable of absorbing its own weight of moisture is a compound capable of absorbing at least fifty times its own weight of moisture.

3. A method according to claim 1, wherein the polyacrylate is sodium polyacrylate.

4. A method according to claim 1, wherein the compound capable of absorbing at least its own weight of moisture is present in an amount of at least 0.1 wt % of the weight of the dry composition.

5. A method according to claim 1, wherein the compound capable of absorbing at least its own weight of moisture is present in an amount of no more than 25 wt % of the weight of the dry composition.

6. A method according to claim 1, wherein the odour-combating zeolitic material is present in an amount at least 0.5 wt % of the weight of the dry composition.

7. A method according to claim 1, wherein the odour-combating zeolitic material is present in an amount of no more than 25 wt % of the weight of the dry composition.

8. A method according to claim 1, wherein the composition further comprises a filler.

9. A method according to claim 9, wherein the filler is an inorganic salt filler.

10. A method according to claim 9, wherein inorganic salt filler is calcium carbonate.

11. A method according to claim 8, wherein the filler is present in the composition in an amount of at least 50 wt % of the weight of the dry composition.

12. A method according to claim 8, wherein the filler is present in the composition in an amount of no more than 99 wt % of the weight of the dry composition.

13. A method according to claim 1, wherein the composition further comprises a fragrance.

14. A method according to claim 1, wherein the chamber is a waste receptacle.

15. A method according to claim 1, wherein the method comprises providing a waste receptacle into which waste material is to be subsequently deposited and adding a layer of the desiccant composition onto the bottom of the substantially enclosed waste receptacle.

16. A method according to claim 1, wherein the method comprises providing a waste receptacle, depositing waste material therein and adding the desiccant composition onto the deposited waste material in the waste receptacle.

17. A composition comprising:
(a) 0.1-25 wt % of a substantially water insoluble polyacrylate compound capable of absorbing at least its own weight of moisture from moist material it is in contact with;
(b) 0.1-25 wt % of an odour-combating zeolitic material;
(c) at least 50 wt % of filler;
the composition being supplied in the form of a flowable powder or granules or a solid composition which falls into a particulate form in use.

18. A product comprising a flowable particulate composition comprising a substantially water insoluble polyacrylate compound capable of absorbing at least its own weight of water, and an odour-combating zeolitic material; the product comprising a container for the composition, the container having a dispenser therefor, the dispenser comprising a plurality of openings to act as a sprinkler head.

19. A product as claimed in claim 18 wherein the openings are such that they are exposed by a user when dispensing is required.

20. (canceled)
